(19) United States
(12) Patent Application Publication
(10) Pub. No.: US 2025/0049289 A1
(43) Pub. Date: Feb. 13, 2025

(54) AEROSOL SANITIZING DEVICE FOR SHOES

(71) Applicant: Dennis Mims, Rio Rancho, NM (US)

(72) Inventor: Dennis Mims, Rio Rancho, NM (US)

(21) Appl. No.: 18/797,455

(22) Filed: Aug. 7, 2024

Related U.S. Application Data
(60) Provisional application No. 63/531,185, filed on Aug. 7, 2023.

(51) Int. Cl. A47L 23/02; A61L 2/18; A61L 2/22; A61L 2/24

(52) U.S. Cl. CPC A47L 23/02; A61L 2/18; A61L 2/22; A61L 2/24; A61L 2202/11; A61L 2202/14; A61L 2202/15; A61L 2202/17

(57) ABSTRACT

An aerosol sanitizing device for shoes comprising a basin defining at least one sanitizing chamber shaped and sized to receive a shoe and operably configured to sanitize and/or disinfect shoe(s) using an aerosol liquid cleaning solution that is dispensed on the shoe(s) when they are in the sanitizing chamber using a conduit network, at least one pump, a plurality of lower liquid aerosol spray nozzles, at least one activation sensor, and an electronic controller. An ultraviolet light-emitting strip disposed on the basin and aimed at the at least one sanitizing chamber further sanitizes and/or disinfects the shoes.

### FIELD OF THE INVENTION

The present invention relates generally to devices to sanitizing and clean shoes and, more particularly, relates to assemblies to house, clean, and sanitizing shoes with an aerosol spray.

### BACKGROUND OF THE INVENTION

Millions of people routinely carry bacteria, including disease pathogens, on the soles of their shoes. This is particularly true in cities and other densely populated areas, where these bacteria are plentiful and easily acquired. Bacteria are then tracked into the home and create a risk of disease. Cleaning the shoe soles of shoes after every excursion from the home could become a tiresome chore. If a device could be provided which quickly and easily sanitizes the shoe soles, such a device would be very useful, and would be well received. An aerosol sanitizing device for shoes, which sprays a quick-drying sanitizing aerosol on the soles of a pair of shoes, would resolve this problem.

Therefore, a need exists to overcome the problems with the prior art as discussed above.

### SUMMARY OF THE INVENTION

The invention provides an aerosol sanitizing device for shoes that overcomes the hereinafore-mentioned disadvantages of the heretofore-known devices and methods of this general type and that includes a basin defining at least one sanitizing chamber, providing a plurality of cleaning apertures, and having a reservoir configured to house a liquid cleaning solution. At least one pump and a conduit network are fluidly coupled to the reservoir. A plurality of lower liquid aerosol spray nozzles are disposed within the plurality of cleaning apertures. At least one activation sensor is operably configured to detect the presence of the shoe within the at least one sanitizing chamber, and an electronic controller communicatively coupled to the at least one activation sensor and the at least one pump cause the at least one pump to transport the liquid cleaning solution from the reservoir through the plurality of lower liquid aerosol spray nozzles and plurality of lower liquid aerosol spray nozzles.

With the foregoing and other objects in view, there is provided, in accordance with the invention, an aerosol sanitizing device, wherein a lower wall defines a plurality of ridges thereon to provide transverse resistance for the shoe.

In accordance with a further feature of the present invention, the lower wall is of an elastomeric material.

In accordance with another feature, the upright orientation angle is approximately 90° relative to the lower wall.

In accordance with yet another feature, in one embodiment of the present invention, the sanitizing chambers consist of two congruent chambers, wherein each congruent chamber is shaped and sized to receive a single shoe.

In accordance with a further feature of the present invention, an embodiment of the present invention further comprises an ultraviolet light-emitting strip horizontally disposed on the basin at a 45° angle aimed at the at least one sanitizing chamber.

In accordance with another feature, the ultraviolet light-emitting strip is disposed inside a triangular-shaped hood disposed on the basin.

In accordance with a further feature of the invention, the least one activation sensors are disposed under each corner of the lower wall.

In accordance with yet another feature, the at least one activation sensors are operably configured to detect the presence of the shoe within the two congruent chambers of the at least one sanitizing chamber and to communicate which of the two congruent chambers contain the shoe to the electronic controller, which causes the at least one pump to transport the liquid cleaning solution from the reservoir to only the plurality of lower liquid aerosol spray nozzles located within the congruent chambers containing the shoe.

Although the invention is illustrated and described herein as embodied in an aerosol sanitizing device for shoes, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not necessarily drawn to scale but, where applicable, may be utilized to support a particular structural configuration or geometric relationship between components utilized in the assembly.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time. Also, for purposes of description herein, the terms “upper”, “lower”, “left,” “rear,” “right,” “front,” “vertical,” “horizontal,” and derivatives thereof relate to the invention as oriented in the figures and is not to be construed as limiting any feature to be a particular orientation, as said orientation may be changed based on the user's perspective of the device. Furthermore, there is no intention to be bound by any expressed or implied theory presented in the preceding technical field, background, brief summary or the following detailed description.

As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. In this document, the term “longitudinal” should be understood to mean in a direction corresponding to an elongated direction of the housing or, where applicable, in the direction from the lower wall of the housing to the upper wall of the housing. The terms “program,” “software application,” and the like as used herein, are defined as a sequence of instructions designed for execution on a computer system. A “program,” “computer program,” or “software application” may include a subroutine, a function, a procedure, an object method, an object implementation, an executable application, an applet, a servlet, a source code, an object code, a shared library/dynamic load library and/or other sequence of instructions designed for execution on a computer system.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 is an elevational front view of an aerosol sanitizing device for shoes, in accordance with the present invention;

FIG. 2 is an elevational side view of the aerosol sanitizing device for shoes according to FIG. 1, in accordance with the present invention;

FIG. 3 is a perspective top view of the aerosol sanitizing device for shoes according to FIG. 1, in accordance with the present invention;

FIG. 4 is an elevational rear view of the aerosol sanitizing device for shoes according to FIG. 1, in accordance with the present invention;

FIG. 5 is a perspective front view of the aerosol sanitizing device for shoes, in accordance with the present invention;

FIG. 6 is a perspective rear view of the aerosol sanitizing device for shoes, in accordance with the present invention;

FIGS. 7-8 depict perspective top views of the aerosol sanitizing device for shoes, in accordance with the present invention;

FIG. 9 is a perspective top view of an exemplary embodiment of the aerosol sanitizing device for shoes, in accordance with the present invention;

FIG. 10 is an exploded view of the aerosol sanitizing device for shoes, in accordance with the present invention;

FIG. 11 is a perspective view of an exemplary embodiment of a reservoir of the aerosol sanitizing device for shoes, in accordance with the present invention;

FIG. 12 is a diagram of a lower wall of the aerosol sanitizing device for shoes having a plurality of ridges thereon, in accordance with the present invention;

FIG. 13 is a cross-sectional view of the aerosol sanitizing device for shoes, in accordance with the present invention;

FIG. 14 is perspective top view of a conduit network of the aerosol sanitizing device for shoes, in accordance with the present invention;

FIG. 15 is a diagram of a perspective top of the aerosol sanitizing device for shoes, in accordance with the present invention; and

FIG. 16 is a block diagram of the electrical components of the aerosol sanitizing device for shoes, in accordance with the present invention.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

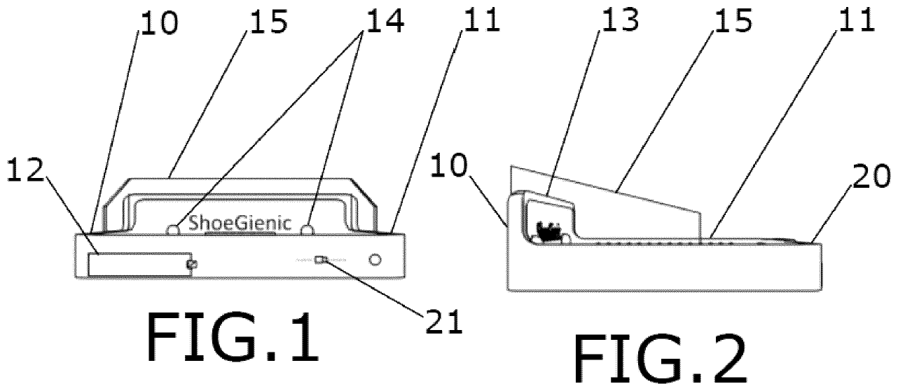

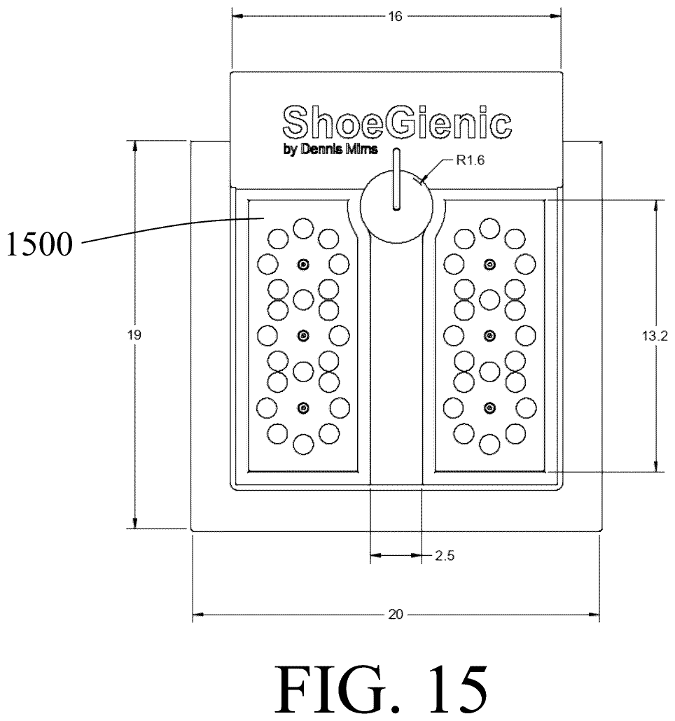

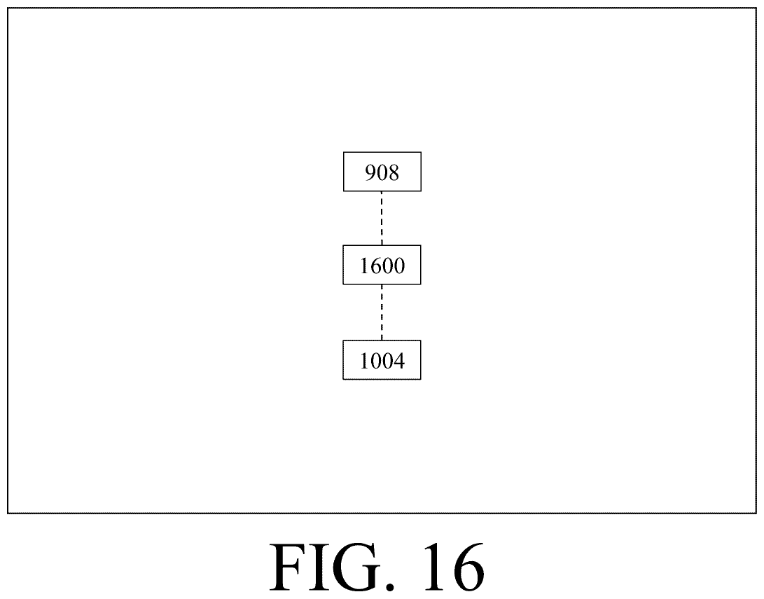

The present invention provides a novel and efficient aerosol sanitizing device for shoes that can quickly and efficiently sanitize shoe(s) using an aerified liquid cleaning solution and/or an ultraviolent disinfecting light. Referring principally now to FIGS. 1-3 and FIGS. 9-10, one embodiment of the present invention is shown. The figures show several advantageous features of the present invention, but, as will be described below, the invention can be provided in several shapes, sizes, combinations of features and components, and varying numbers and functions of the components. The first example of a device for cleaning shoes (hereinafter, “device 100” or “device 10”) includes a basin 900 (also referred to as “basin 11”), which may also be interchangeably referred to herein as a housing 900 or housing 11. The basin 900 defines at least one sanitizing chamber 902 shaped and sized to receive a shoe. In embodiments with only a single sanitizing chamber 902, exemplary dimensions of the sanitizing chamber 902 are approximately 20 inches in length and 19 inches in width, as depicted in FIG. 15, to accommodate various shaped and sized feet and shoes but still maintain a relatively close distance between the shoe and the walls of the basin 900. Too much space or distance between the shoe and the walls of the basin 900 could compromise the sanitizing and/or disinfecting efficacy of the device 100. The dimensions of the device 100, therefore, are significant though they may have slight variations. In embodiments with two sanitizing chambers 902a-b, the foregoing exemplary length of each would be approximately half, e.g., each of the two sanitizing chambers 902a-b would be approximately 8 to 10 inches in length depending on whether, and how wide of, a dividing physical barrier or space is placed between the two sanitizing chambers 902a-b.

The basin 900 further has a lower wall 1500 (also referred to as “lower wall 22”) defining the at least one sanitizing chamber 902 and defining a plurality of lower cleaning apertures 904a-n, and having a sidewall 906 (also referred to as “sidewall 24”) surrounding the lower wall 1500 and defining a plurality of side cleaning apertures 26a-n (wherein “n” refers to any number greater than one). As used herein, the term “wall” is intended broadly to encompass continuous structures, as well as, separate structures that are coupled together so as to form a substantially continuous external surface.

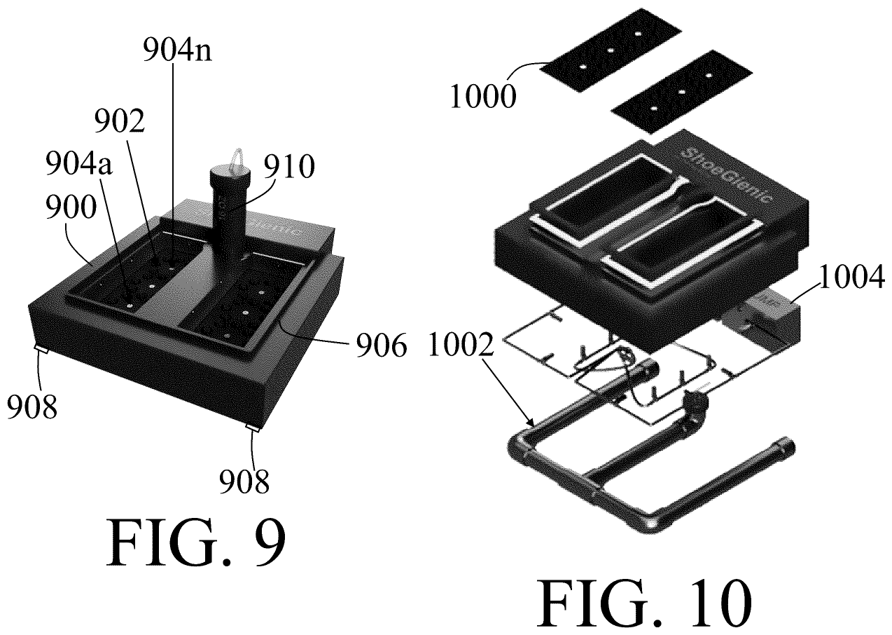

As best depicted in FIG. 9 and FIG. 11, the device 100 also comprises a reservoir 910 (also referred to as “reservoir 20”) configured to house a liquid cleaning solution that cleans, rinses, sanitizes, and/or disinfects. In an exemplary embodiment, the reservoir 910 has a removable lid to conveniently access and refill the reservoir, and has a volume of approximately 16 fluid ounces. While the size and volume of the reservoir 910 may vary, it is beneficially large enough to hold enough of the liquid cleaning solution to accommodate multiple uses of the device 100 before a refill becomes necessary while being compact enough to fit within the device 100. In an exemplary embodiment, the reservoir 910 is approximately 3.2 inches wide, corresponding to the diameter of a 16 ounce bottle of water.

At least one pump 1004 is fluidly couplable to the reservoir 910 and a conduit network 1000 is fluidly coupled to the reservoir 910 and the at least one pump 1004 and disposed within the basin 900. The at least one pump 1004 and the conduit network 1000 are best reflected in FIG. 14. In one exemplary embodiment, the at least one pump 1004 has dimensions of 7 inches by 4 inches by 3.2 inches, ⅜ inches (9.5 mm) Barbed; power of 12V, 4.5 A; a flow rate of 1.3 GPM at open flow; and a pressure of 100 psi/7.08 bar. Specific nozzles and other connectors are subject to minor changes.

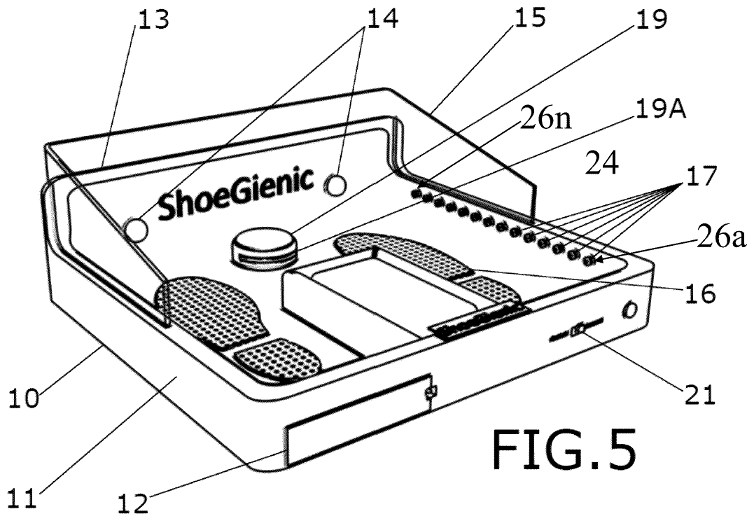

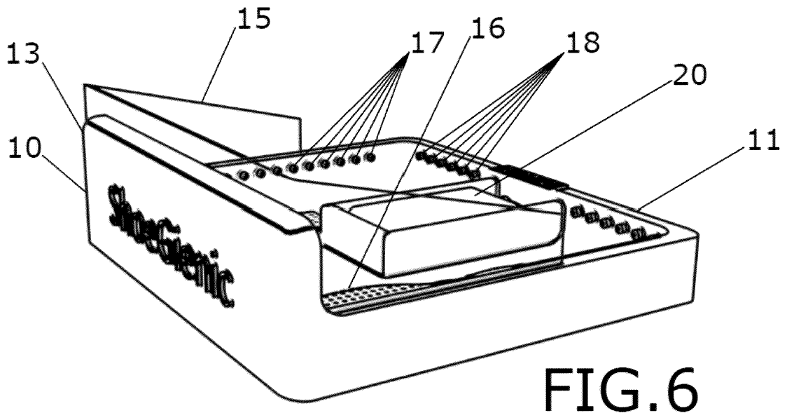

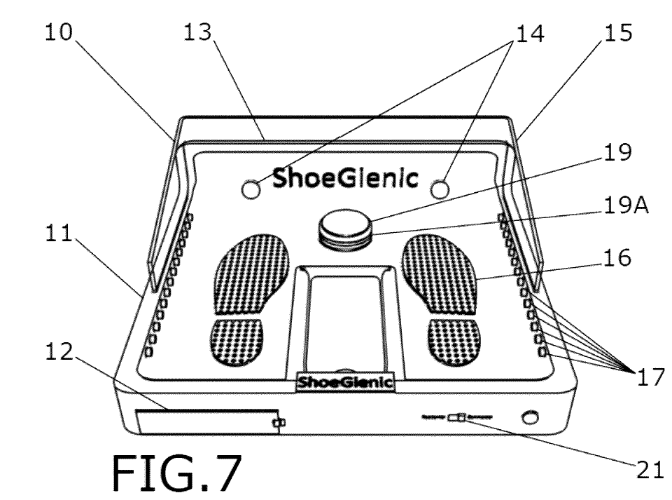

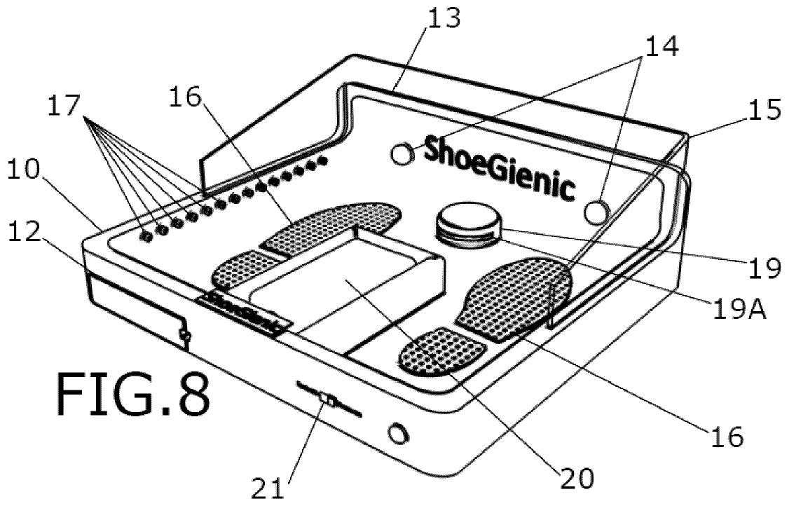

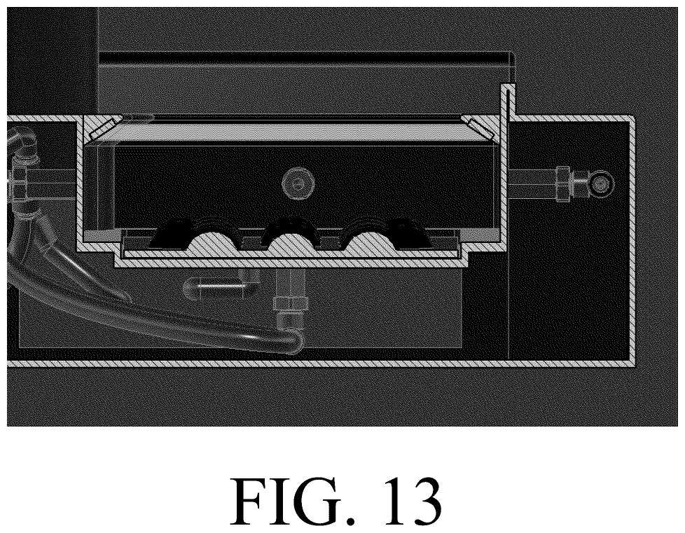

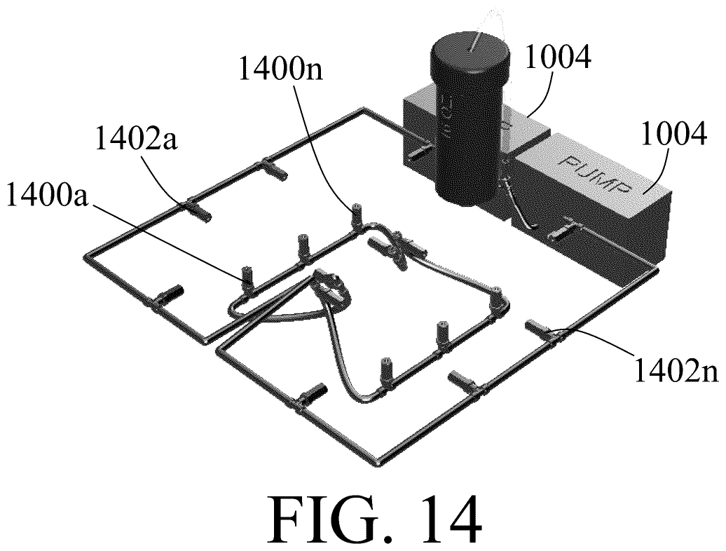

Exemplary embodiments have two independent pumps 1004a-b capable of actuating each sanitizing chamber 902a-b independently. Consequently, if only one shoe is placed in one sanitizing chamber 902a, only that sanitizing chamber 902a will be activated and supplied with the liquid cleaning solution by the pump 1004a corresponding to that specific sanitizing chamber 902a. This will be sensed via at least one activation sensor 908 (also referred to as “activation sensor 14”) operably configured to detect the presence of the shoe within the at least one sanitizing chamber 902. In one exemplary embodiment depicted in FIGS. 1-8, the at least one activation sensor 908 is in the form of a laser detector or laser detection system. In a second exemplary embodiment depicted in FIGS. 9-14, the at least one activation sensor 908 is in the form of piezoelectric load sensors which are thin and robust sensors (approximately 0.014 in thickness) that can be placed under the housing 900, right under the supports bearing the load of the user. When the user stands in the at least one sanitizing chamber 902, a load is sensed by the activation sensor 908 and the pumps 1004a-n are actuated to start disinfecting operation. This can be programmed so depending on which foot has been placed, only one side will activate. The activation sensors 908 are disposed on the rear panel 12, which detects the presence of shoes or similar objects resting in the at least one sanitizing chamber 902.

The device 100 further comprises a plurality of lower liquid aerosol spray nozzles 1400a-n (also referred to herein as “spray areas 16” or “lower liquid aerosol spray nozzles 16”) disposed within the plurality of lower cleaning apertures 904a-n and angled at least partially in an upright orientation angle relative to the lower wall 1500 to spray within the at least one sanitizing chamber 902. A plurality of lower liquid aerosol spray nozzles 1402a-n (also referred to as “lower liquid aerosol spray nozzles 17 and/or 18”) are disposed within the plurality of side cleaning apertures 26a-n and angled at an acute angle orientation relative to the upright orientation angle of the plurality of lower liquid aerosol spray nozzles 1400a-n. An electronic controller 1600 is communicatively coupled to the at least one activation sensor 908 and the at least one pump 1004 and cause the at least one pump 1004 to transport the liquid cleaning solution from the reservoir 910 through the plurality of lower liquid aerosol spray nozzles 1400a-n and plurality of lower liquid aerosol spray nozzles 1402a-n.

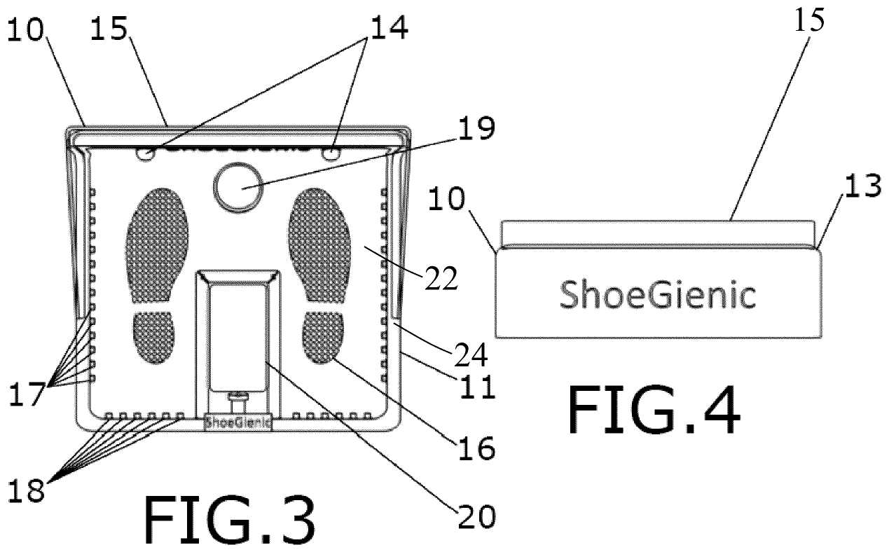

In one embodiment (depicted in FIGS. 3-8), a large button 19 is provided between the plurality of lower liquid aerosol spray nozzles 1400a-n. If the button 19 is pressed, after a brief predetermined delay period such as one second, the device 100 sprays a quick-drying sanitizing aerosol upward through the apertures of the plurality of lower liquid aerosol spray nozzles 1400a-n, and out through the plurality of lower liquid aerosol spray nozzles 1402a-n. The button 19 may be beneficially illuminated in red when activated, then in green when the spraying is completed.

The device 100 is battery-powered and provides a battery door 12 on the exterior front surface to enable the user to change the batteries. A transparent shield 15, extending upward from the rear panel 13, is provided to prevent the aerosol fluid from escaping the basin 11.

The device 10 further provides a selector switch 21 on the front surface of the basin 900, which toggles between a commercial setting and a personal setting. When the personal setting is selected, the side sprayers 17 are configured with a program or controller to be inoperative, thereby preserving sanitizing liquid in the reservoir 20.

To use the first exemplary embodiment, the user may step into the basin 11 such that the soles of the user's shoes are within the spray areas 16. The user may then tap the button 18 with a toe, and return the foot to the spray area 16. After a brief, predetermined delay period, such as one second, the device 10 sprays a quick-drying sanitizing aerosol upward through the apertures of the spray areas 16, and out through the side sprayers 17 and the rear sprayers 18.

The basin 11, the battery door 12, the rear panel 13, the sensors 14, the shield 15, the spray areas 16, the side sprayers 17, the rear sprayers 18, the button 19, and the fluid reservoir 20 are preferably manufactured from a rigid, durable material with substantial structural strength, such as plastic, brass, or aluminum alloy. Components and methods listed above are preferable, but artisans will recognize that alternate components and methods could be selected without altering the scope of the invention.

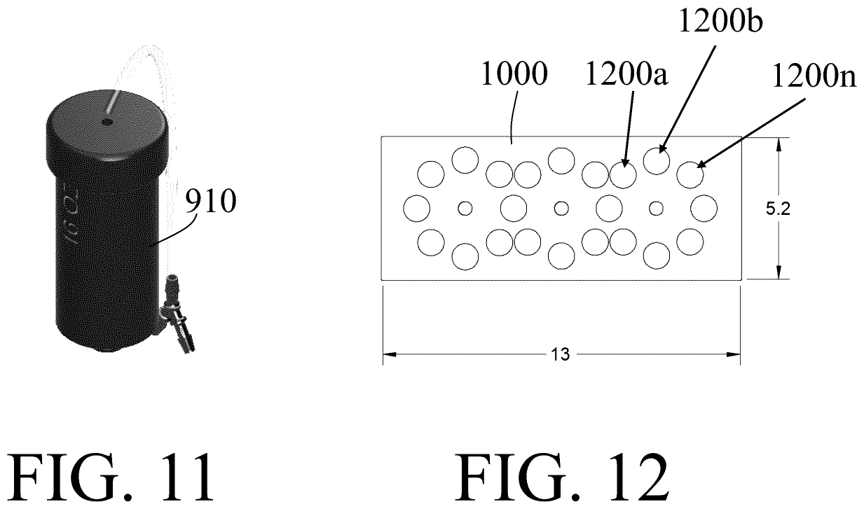

As best depicted in FIG. 12, the lower wall 1500 may define a plurality of ridges 1200a-n thereon to provide transverse (wherein “transverse” is defined as “from side to side of the sidewall”) resistance for the shoe. Beneficially, the surface area for the sanitizing solution is maximized via the application of the plurality of ridges 1200a-n in the form of spherical pegs which have minimal surface area contact with the sole of the shoe. The lower wall 1500 may also be of an elastomeric material, such as silicon, to facilitate movement of the shoe across the lower wall 1500 and to facilitate a better cleaning and sanitation of the shoe.

In accordance with an exemplary embodiment, the upright orientation angle is approximately 90° (+/−15°) relative to the lower wall 1500, to accommodate an appropriate and comfortable fit of the shoe in the sanitizing chambers 902. The sanitizing chambers 902 may consist of two congruent (defined as of identical size and of corresponding shape, e.g., mirror-images of one another) chambers, wherein each congruent chamber is shaped and sized to receive a single shoe.

An ultraviolet light-emitting strip horizontally is disposed on the basin at a 45° (+/−15°) angle aimed at the at least one sanitizing chamber 902, to ensure the sanitizing ultraviolet (“UV”) light is directed at the shoes. A triangular shaped hood disposed on the basin 900 houses the UV strip and serves as an extra layer of protection for the disinfectant to remain within the disinfection volume.

In one embodiment, the least one activation sensors 908 are disposed under each corner of the lower wall 1500 to efficiently detect and/or measure the load in the basin 900 and prompt the pump 1004 to transport the liquid cleaning solution from the reservoir 910 through the plurality of lower liquid aerosol spray nozzles 1400a-n and plurality of lower liquid aerosol spray nozzles 1402a-n. The at least one activation sensors 908 are operably configured to detect the presence of the shoe within the two congruent chambers of the at least one sanitizing chamber 902 and to communicate which of the two congruent chambers contain the shoe to the electronic controller 1600, which causes the at least one pump 1004 to transport the liquid cleaning solution from the reservoir 910 to only the plurality of lower liquid aerosol spray nozzles located within the congruent chambers containing the shoe

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the above described features.

What is claimed is:

1. An aerosol sanitizing device for shoes comprising:
a basin defining at least one sanitizing chamber shaped and sized to receive a shoe, having a lower wall defining the at least one sanitizing chamber and defining a plurality of lower cleaning apertures, and having a sidewall surrounding the lower wall and defining a plurality of side cleaning apertures;
a reservoir configured to house a liquid cleaning solution;
at least one pump fluidly couplable to the reservoir;
a conduit network fluidly coupled to the reservoir and the at least one pump and disposed within the basin;
a plurality of lower liquid aerosol spray nozzles disposed within the plurality of lower cleaning apertures and angled at least partially in an upright orientation angle relative to the lower wall to spray within the at least one sanitizing chamber;
a plurality of lower liquid aerosol spray nozzles disposed within the plurality of side cleaning apertures and angled at an acute angle orientation relative to the upright orientation angle of the plurality of lower liquid aerosol spray nozzles;
at least one activation sensor operably configured to detect the presence of the shoe within the at least one sanitizing chamber; and
an electronic controller communicatively coupled to the at least one activation sensor and the at least one pump and cause the at least one pump to transport the liquid cleaning solution from the reservoir through the plurality of lower liquid aerosol spray nozzles and plurality of lower liquid aerosol spray nozzles.

2. The aerosol sanitizing device according to claim 1, wherein the lower wall defines a plurality of ridges thereon to provide transverse resistance for the shoe.

3. The aerosol sanitizing device according to claim 2, wherein the lower wall is of an elastomeric material.

4. The aerosol sanitizing device according to claim 1, wherein the upright orientation angle is approximately 90° relative to the lower wall.

5. The aerosol sanitizing device according to claim 1, wherein the sanitizing chambers consist of two congruent chambers, wherein each congruent chamber is shaped and sized to receive a single shoe.

6. The aerosol sanitizing device according to claim 1, further comprising an ultraviolet light-emitting strip horizontally disposed on the basin at a 45° angle aimed at the at least one sanitizing chamber.

7. The aerosol sanitizing device according to claim 6, wherein the ultraviolet light-emitting strip is disposed inside a triangular-shaped hood disposed on the basin.

8. The aerosol sanitizing device according to claim 1, wherein the least one activation sensors are disposed under each corner of the lower wall.

9. The aerosol sanitizing device according to claim 5, wherein the at least one activation sensors are operably configured to detect the presence of the shoe within the two congruent chambers of the at least one sanitizing chamber and to communicate which of the two congruent chambers contain the shoe to the electronic controller, which causes the at least one pump to transport the liquid cleaning solution from the reservoir to only the plurality of lower liquid aerosol spray nozzles located within the congruent chambers containing the shoe.
